(19) United States
(12) Patent Application Publication
(10) Pub. No.: US 2025/0331786 A1
(43) Pub. Date: Oct. 30, 2025

(54) LIFTING PLATFORM AND MEDICAL DEVICE

(71) Applicant: GE Precision Healthcare LLC, Waukesha, WI (US)

(72) Inventors: Ling Zhang, Beijing (CN); Yigang Xia, Beijing (CN); Yanan Lyu, Beijing (CN); Kun Zhao, Beijing (CN)

(21) Appl. No.: 19/195,114

(22) Filed: Apr. 30, 2025

(30) Foreign Application Priority Data
Apr. 30, 2024 (CN) 202420941913.2

(51) Int. Cl. A61B 6/04; A61B 6/03

(52) U.S. Cl. CPC A61B 6/0407; A61B 6/03; A61B 6/0487

(57) ABSTRACT

Provided in embodiments of the present application are a lifting platform and a medical device. The lifting platform includes: a base; a support table disposed on an upper side of the base; a support assembly connecting the support table and the base and driving the support table to lift; a locking assembly including a body member connected to the support assembly and a blocking member movably mounted on the body member, wherein the blocking member prevents movement of the support assembly when moving to a first position abutting against the base; and a lock status switching member detachably connected to the locking assembly, wherein the blocking member moves to the first position when the lock status switching member is removed from the locking assembly.

### CROSS-REFERENCE TO RELATED APPLICATIONS

This application claims priority to Chinese Application No. 202420941913.2, filed on Apr. 30, 2024, now Chinese Patent No. ZL202420941913.2, the disclosure of which is incorporated herein by reference in its entirety.

### TECHNICAL FIELD

Embodiments of the present application relate to the technical field of medical devices, and in particular to a lifting platform and a medical device.

### BACKGROUND

Lifting platforms are widely used in medical devices. Taking an example in which the medical device is an medical imaging system, radiation from a ray source is emitted toward a subject under examination, and the subject under examination is usually a patient in a medical diagnosis application. A part of the radiation is received by a detector through the subject under examination, and an output signal is generated based on the amount or intensity of the radiation received by the detector. Related processing is performed on the output information to generate a medical image that can be displayed for viewing.

For a lying-position scanning mode, typically, the subject under examination needs to lie on a lifting platform in a lying position. The lifting platform includes a base and a support table disposed on an upper side of the base. A support assembly is disposed between the base and the support table, and the support assembly drives the support table to move, so that the subject under examination placed on the support table is moved to an expected position.

### SUMMARY

The inventor has found that when a user debugs or maintains a lifting platform, in order to avoid unexpected displacement or lifting of a support table, some locking apparatuses usually need to be installed in the lifting platform to support or fasten the support table. However, such a design requires the user to install additional locking apparatuses in the lifting platform to support or fasten the support table, and if the locking apparatuses are lost or no suitable locking apparatus can be found, it is impossible to perform reliable locking. In addition, if the user forgets to install the locking apparatus, the support table may suddenly drop, which easily causes a safety problem or even serious injury.

In view of at least one of the above technical problems, embodiments of the present application provide a lifting platform and a medical device.

According to one aspect of the embodiments of the present application, provided is a lifting platform. The lifting platform includes a base; a support table disposed on an upper side of the base; a support assembly connecting the support table and the base and driving the support table to lift; a locking assembly comprising a body member connected to the support assembly and a blocking member movably mounted on the body member, wherein the blocking member prevents movement of the support assembly when moving to a first position abutting against the base; and a lock status switching member detachably connected to the locking assembly, wherein the blocking member moves to the first position when the lock status switching member is removed from the locking assembly.

According to another aspect of the embodiments of the present application, the lifting platform further includes a shielding member for a component to be repaired covering or shielding at least a part of a component to be repaired of the lifting platform, wherein the lock status switching member is configured to release covering or shielding of the component to be repaired by the shielding member for a component to be repaired.

According to another aspect of the embodiments of the present application, the lifting platform further includes a fastening member connected to the shielding member for a component to be repaired to fasten the shielding member for a component to be repaired to a position covering or shielding at least the part of the component to be repaired, wherein the lock status switching member is configured to release the connection between the fastening member and the shielding member for a component to be repaired.

According to another aspect of the embodiment of the present application, the lock status switching member comprises a first lock status switching member and a second lock status switching member, the fastening member comprises a first fastening member and a second fastening member, the first lock status switching member is configured to release a connection between the first fastening member and the shielding member for a component to be repaired, and the second lock status switching member is configured to release a connection between the second fastening member and the shielding member for a component to be repaired.

According to another aspect of the embodiments of the present application, the blocking member comprises a first blocking member and a second blocking member, the first blocking member abuts against the base when the first lock status switching member is removed from the locking assembly, and the second blocking member abuts against the base when the second lock status switching member is removed from the locking assembly.

According to another aspect of the embodiments of the present application, a through hole is disposed in the body member, and the through hole extends in a moving direction of the blocking member for accommodating at least a part of the blocking member.

According to another aspect of the embodiments of the present application, the locking assembly further includes an elastic member that is accommodated in the through hole and that applies a force to the blocking member to move the blocking member toward the base.

According to another aspect of the embodiments of the present application, the elastic member is disposed inside the blocking member.

According to another aspect of the embodiments of the present application, a first hole portion is disposed on a first end that is of the blocking member and that is away from the base, the locking assembly further comprises a guide member, one end of the guide member is fixedly connected to the body member, the other end of the guide member is located in the first hole portion, and the blocking member moves along the guide member; and the elastic member is accommodated in the first hole portion, one end of the elastic member abuts against the bottom of the first hole portion, and the other end of the elastic member abuts against the guide member.

According to another aspect of the embodiments of the present application, when the lock status switching member is connected to the locking assembly, the blocking member moves to a second position that does not abut against the base.

According to another aspect of the embodiments of the present application, when the lock status switching member is connected to the locking assembly, the lock status switching member is engaged with the blocking member.

According to another aspect of the embodiments of the present application, the blocking member comprises an engagement portion that is engaged with the lock status switching member, and the engagement portion is a groove disposed on a surface of the blocking member or a hole portion disposed in the blocking member.

According to another aspect of the embodiments of the present application, a second hole portion is disposed in the body member, and the second hole portion extends in a direction different from a moving direction of the blocking member for accommodating the lock status switching member; and when the lock status switching member is located in the second hole portion, and the blocking member is located at a second position, the lock status switching member is engaged with the blocking member.

According to another aspect of the embodiments of the present application, when the lock status switching member abuts against an end portion that is of the body member and that is away from the base, and the blocking member is located at a second position, the lock status switching member is engaged with the blocking member.

According to another aspect of the embodiments of the present application, a medical device is provided. The medical device comprises the lifting platform mentioned above.

One of the beneficial effects of the embodiments of the present application is that: a locking assembly capable of preventing movement of a support assembly of a lifting platform, and a lock status switching member detachably connected to the locking assembly are disposed in the lifting platform, and the locking assembly prevents the movement of the support assembly when the lock status switching member is removed from the locking assembly. Therefore, a user only needs to remove the lock status switching member from the locking assembly to lock the lifting platform. This can simplify the locking operation, and help avoid safety problems.

With reference to the following description and drawings, specific implementations of the embodiments of the present application are disclosed in detail, and the way in which the principles of the embodiments of the present application can be employed are illustrated. It should be understood that the implementations of the present application are not limited in scope thereby. Within the scope of the spirit and clauses of the appended claims, the implementations of the present application include many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and those of ordinary skill in the art may obtain other implementations according to the drawings without involving inventive effort. In the drawings:

FIG. 1 is a schematic diagram of a CT system for CT imaging according to an embodiment of the present application;

FIG. 2 is another schematic diagram of a CT system for CT imaging according to an embodiment of the present application;

FIG. 3 is a schematic diagram of a lifting platform according to an embodiment of the present application;

FIG. 4 is a schematic diagram of a local structure of a lifting platform according to an embodiment of the present application;

FIG. 5 is another schematic diagram of a local structure of a lifting platform according to an embodiment of the present application;

FIG. 6a, FIG. 6b, and FIG. 9 are schematic diagrams of a locking assembly and a lock status switching member according to embodiments of the present application; and

FIG. 7, FIG. 8, and FIG. 10 are additional schematic diagrams of a locking assembly according to embodiments of the present application.

### DETAILED DESCRIPTION

Specific implementations of the present application are described below. It should be noted that in the specific description of said implementations, for the sake of brevity and conciseness, the present description cannot describe all of the features of the actual implementations in detail. It should be understood that in the actual implementation process of any implementation, just as in the process of any one engineering project or design project, a variety of specific decisions are often made to achieve specific goals of the developer and to meet system-related or business-related constraints, which may also vary from one implementation to another. Furthermore, it should also be understood that although efforts made in such development processes may be complex and tedious, for those of ordinary skill in the art related to the content disclosed in the present application, some design, manufacture, or production changes made on the basis of the technical content disclosed in the present application are only common technical means, and should not be construed as the content of the present application being insufficient.

Unless defined otherwise, the technical terms or scientific terms used in the claims and the description should have the usual meanings that are understood by those of ordinary skill in the technical field to which the present application belongs. Terms such as “first”, “second”, and similar terms used in the description and claims of the patent application of the present application do not denote any order, quantity, or importance, but are merely intended to distinguish between different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects. The terms “connect” or “link” and similar words are not limited to physical or mechanical connections, and are not limited to direct or indirect connections.

In embodiments of the present application, a lifting platform may be used in various devices. For example, the device includes a medical device, an industrial device, or the like. Taking the medical device as an example, for example, the medical device includes devices related to various medical imaging scenarios. The medical imaging scenarios include but are not limited to magnetic resonance imaging (MRI), computed tomography (CT), ultrasound imaging, X-ray imaging, positron emission computed tomography (PET), single photon emission computed tomography (SPECT), PET/CT, PET/MR, or any other suitable medical imaging scenario.

In the embodiments of the present application, the CT scenario is taken as an example to describe the lifting platform and the medical device of the present application. Those skilled in the art will understand that the present application is also applicable to other radiographic imaging systems or imaging systems based on other high-frequency electromagnetic energy.

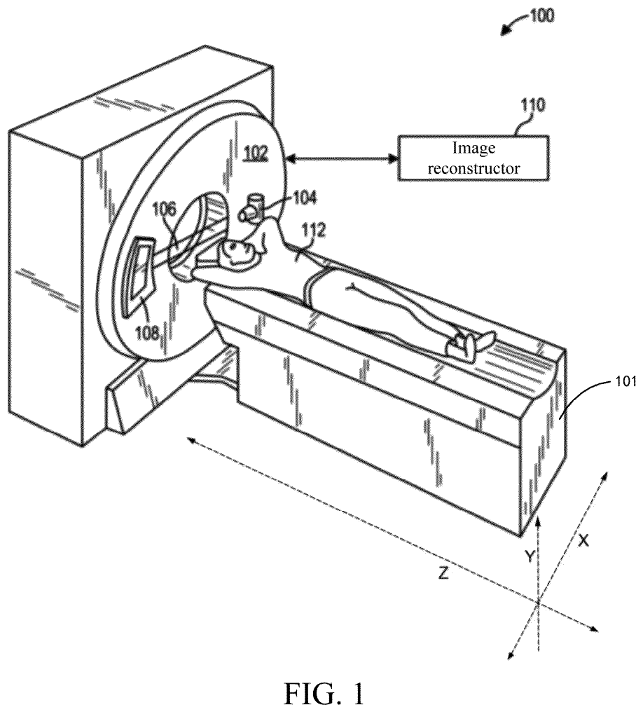

FIG. 1 is a schematic diagram of a CT system for CT imaging according to an embodiment of the present application. As shown in FIG. 1, a CT system 100 is configured to image a scan subject 112 (such as a patient, an inanimate object, or an implant, a stent, and/or a contrast agent present in a body).

The CT system 100 includes a lifting platform 101. The scan subject 112 lies on the lifting platform 101, and the lifting platform 101 drives the subject 112 to be displaced, for example, displaced along at least one of the coordinate axes X, Y, and Z in FIG. 1.

The CT system 100 includes a gantry 102. An X-ray source 104 is mounted on the gantry 102, and the X-ray source 104 is configured to emit an X-ray beam 106 for imaging the scan subject 112. Specifically, the X-ray source 104 is configured to project the X-ray beam 106 toward a detector assembly 108 positioned on the opposite side of the gantry 102.

In some embodiments, the X-ray source 104 emits a fan-shaped/cone-shaped ray beam that is collimated to be located in an X-Y-Z plane, namely, an imaging plane, of a

Cartesian coordinate system. The X-ray beam passes through the scan subject 112 and is attenuated by the scan subject 112 before being projected into the detector assembly 108.

Therefore, the intensity of the ray received on the detector assembly 108 depends on the degree of attenuation of the ray by the scan subject 112. The detector assembly 108 may include a plurality of detector units forming an array, and each detector unit detects a signal indicating the intensity of a corresponding ray beam. The signal detected by each detector unit may be provided as distributed projection data (or image data) for subsequent processing.

In some embodiments, the CT system 100 further includes an image reconstructor 110 for receiving projection data and performing image reconstruction to obtain a volumetric image of the scan subject 112, which may specifically include a three-dimensional volumetric image or an image corresponding to one or a plurality of two-dimensional slices of the scan subject 112. The reconstructed image may be transmitted to a processing device and stored in a mass memory coupled to the processing device.

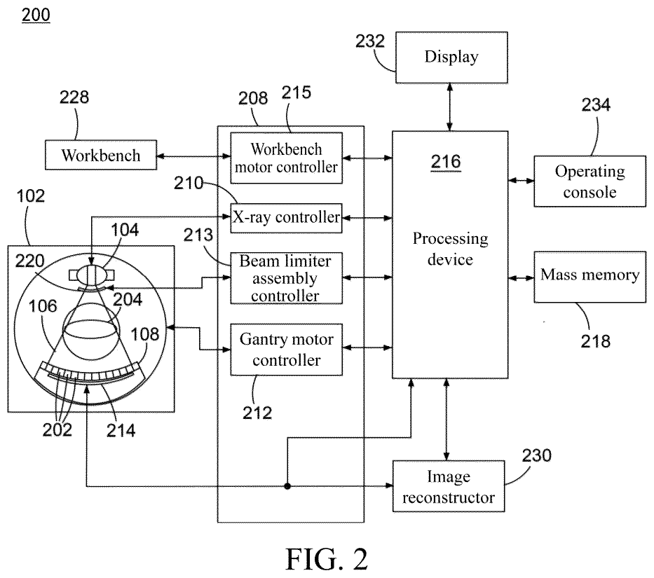

FIG. 2 is another schematic diagram of a CT system for CT imaging according to an embodiment of the present application. A CT system 200 shown in FIG. 2 is similar to the CT system 100 shown in FIG. 1. As shown in FIG. 2, the CT system 200 is configured to image a scan subject 204 (e.g., the scan subject 112 in FIG. 1).

The CT system 200 includes a detector assembly 108, and the detector assembly includes a plurality of detector units 202 that jointly detect an X-ray beam 106 passing through the scan subject 204 to acquire corresponding projection data. In an example, the detector assembly includes a plurality of rows of detector units 202 that may be configured to acquire projection data corresponding to a plurality of slices. The detector unit 202 may be an energy integrating detector, a photon counting detector, an energy discriminating detector, or any other suitable radiation detector. The detector unit 202 may use a scintillator to generate an analog signal, and the detector unit 202 may alternatively be a direct conversion detector (that is, a detector that does not use a scintillator), such as a detector based on a silicon strip or another semiconductor material. When a semiconductor sensor is exposed to the X-ray, the detector generates a measurable signal.

In some embodiments, the CT system 200 further includes a beam limiter assembly 220. The beam limiter assembly 220 may be positioned in a propagation direction of the X-ray source 104 and adjacent to the X-ray source, and the beam limiter assembly 220 may define the shape and/or the range of a high-intensity region of the X-ray beam 106 to limit X-ray exposure at a position outside the region of interest of the scan subject 204. In particular, the beam limiter assembly 220 may be mounted between the X-ray source 104 and the scan subject 204, and positioned near the X-ray source 104. The beam limiter assembly 220 may include one or a plurality of blades (or baffles). The one or the plurality of blades forms/form an opening, the one or the plurality of blades is/are configured to block one part of the X-ray, and the opening is used for passage of the other part of the ray. The shape and/or the range of the projected X-ray beam are/is defined in such a way.

In some embodiments, the CT system 200 may further include a collimator (not shown in the figure) that may be disposed between the scan subject 204 and the detector assembly 208 and adjacent to the detector assembly 208. The collimator is typically made of a material that blocks or absorbs an X-ray. Therefore, an X-ray reflected or deflected via the scan subject 204 can be blocked by the collimator, to prevent the X-ray from being projected into the detector assembly 208. On the contrary, the X-ray 106 traveling in a relatively straight path from the X-ray source 104 to the detector assembly 208 is not blocked by the collimator 222 and can be projected into the detector assembly 208.

In some embodiments, the CT system 200 further includes a control mechanism 208, and the control mechanism 208 may include an X-ray controller 210 and a gantry motor controller 212. During scanning for acquiring projection data, the X-ray controller 210 is configured to control an operation of the X-ray source 104 (e.g., providing power and timing signals to the X-ray source 104 and determining/tracking a focus/focal spot position of the X-ray source), and the gantry motor controller 212 is configured to control, based on an imaging requirement, the gantry 102 and components mounted thereon to rotate at a suitable speed around the scan subject 112, so that an angle/a position of intersection between the X-ray beam 106 and the scan subject 112 is continuously changed. Projection data acquired at each angle by the detector assembly 108 is referred to as a view, and the scanning performed on the scan subject 112 includes a set of views obtained at different angles during one rotation of the X-ray source 104 and the detector assembly 108 around the scan subject 112.

In some embodiments, the control mechanism 208 further includes a lifting platform motor controller 215, and the lifting platform motor controller 215 may further control a lifting platform 228 (e.g., the scanning lifting platform 101 in FIG. 1). In particular, the lifting platform motor controller 215 may move the lifting platform 228, so that the scan subject 204 on the lifting platform 228 is moved completely or partially through the opening of the gantry 102 in FIG. 1, to position the scan subject 204 in a scanning region defined by the gantry 102, so as to acquire projection data corresponding to the scan subject 204.

In some embodiments, the control mechanism 208 further includes a beam limiter assembly controller 213 configured to control an operation of the beam limiter assembly 220, for example, control movement of the one or the plurality of blades of the beam limiter assembly 220 (or a beam limiter assembly 300 to be described below) to adjust the position or the width of the opening. In some embodiments, such an adjustment may be performed based on a result of tracking the focus of the X-ray source by the X-ray controller 210.

The detector array consisting of the detector units 202 includes an umbra region for receiving umbra data and a penumbra region for receiving penumbra data. When the X-ray source 104 emits the X-ray beam 106 at different focal spot positions, the umbra region is capable of receiving X-ray beams at all focal spot positions, and the penumbra region is capable of receiving only X-ray beams emitted at some of the focal spot positions. In some embodiments, the umbra region is generally formed into a regular array (such as a rectangle) to accommodate the shape of the X-ray beam defined via, for example, the beam limiter assembly 220 having a square opening. In order to track the focal spot position, a small quantity of additional detectors need to be disposed at an edge portion outside the regular array to form the penumbra region mentioned above. Therefore, an irregular shape is formed as a whole, resulting in high design costs.

In some embodiments, the CT system 200 further includes a data acquisition system (DAS) 214 configured to sample and digitize the data received by the detector unit 202 to convert the data into projection data. As further described herein, the data sampled and digitized by the DAS 214 is transmitted to a processing device 216.

The processing device 216 may include a computer device. In an example, the processing device 216 stores data in a storage device or a mass memory 218, or the data sampled and digitized by the DAS 214 is transmitted directly to the mass memory 218. For example, the storage device 218 may include a hard disk drive, a floppy disk drive, a compact disc-read/write (CD-R/W) drive, a digital versatile disc (DVD) drive, a flash drive, and/or a solid-state storage drive. An associated display 232 allows an operator to observe the reconstructed image and other data from the processing device. The display 232 may also allow the operator to select a region of interest and/or request patient information via a graphical user interface for subsequent processing.

In some embodiments, the CT system 200 further includes an operating console 234 that is operatively coupled to the processing device 216, and the processing device 216 may receive instructions and parameters input by the operator through the operating console 234. The operating console has an operator interface in a certain form, such as a keyboard, a mouse, a voice activated controller, or any other suitable input apparatus.

In addition, the processing device 216 is configured to send commands or parameters to one or more of the DAS 214, the X-ray controller 210, the gantry motor controller 212, the beam limiter assembly controller 213, and the lifting platform motor controller 215 to control a system operation, such as data acquisition and/or processing. The processing device 216 may control the system operation based on an operator input.

In some embodiments, the processing device 216 may calculate, based on the penumbra data and the umbra data in the data received from the DAS, a parameter (e.g., a Z ratio) used to track the focus of the X-ray source 104, and send the parameter to the X-ray controller 210, and the X-ray controller 210 tracks the focus position of the X-ray source 104 based on the received parameter (e.g., the Z ratio). The processing device 216 may further receive a result of focus tracking from the X-ray controller 210 and send the result to the beam limiter assembly controller 213 to be able to control an operation of the beam limiter assembly 220 (or the beam limiter assembly 300) based on the current focus position.

In some embodiments, the CT system 200 includes an image reconstructor 230, the image reconstructor 230 is configured to receive the projection data from the DAS 214 and perform image reconstruction, and the reconstructed image may be transmitted to the processing device 216 to generate patient information for diagnosis and evaluation. The processing device 216 is configured to store the reconstructed image and the patient information in the mass memory 218. In some embodiments, the processing device 216 may transmit the reconstructed image to the display 232, and the display 232 may be communicatively coupled to the processing device 216 and/or the image reconstructor 230.

Only one example of a CT system suitable for applying the embodiments of the present application is described above. A person skilled in the art will recognize that the embodiments of the present application are also applicable to any imaging system or device based on other radiographic rays or high-frequency electromagnetic energy.

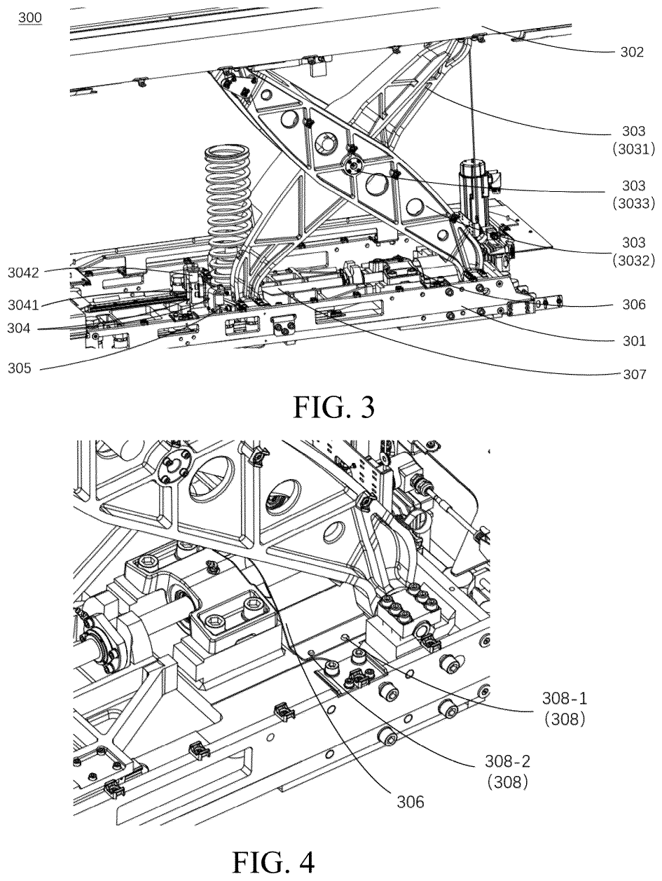

FIG. 3 is a schematic diagram of a lifting platform according to an embodiment of the present application. As shown in FIG. 3, a lifting platform 300 includes a base 301, a support table 302, a support assembly 303, a locking assembly 304, and a lock status switching member 305.

As shown in FIG. 3, the support table 302 is disposed on an upper side of the base 301. The support assembly 303 connects the support table 302 and the base 301, and drives the support table 302 to lift.

The locking assembly 304 includes a body member 3041 connected to the support assembly 303 and a blocking member 3042 movably mounted on the body member 3041. The blocking member 3042 prevents movement of the support assembly 303 when moving to a first position abutting against the base 301.

The lock status switching member 305 is detachably connected to the locking assembly 304. The blocking member 3042 moves to the first position when the lock status switching member 305 is removed from the locking assembly 304.

According to the foregoing embodiment, the locking assembly 304 capable of preventing the movement of the support assembly 303 of the lifting platform 300, and the lock status switching member 305 detachably connected to the locking assembly 304 are disposed in the lifting platform 300, and the locking assembly 305 prevents the movement of the support assembly 303 when the lock status switching member 304 is removed from the locking assembly 304. Therefore, a user only needs to remove the lock status switching member 305 from the locking assembly 304 to lock the lifting platform 300, that is, the lifting platform 300 is locked by a removing operation instead of a mounting operation. Therefore, this can simplify the locking operation, and help avoid safety problems.

In some embodiments, as shown in FIG. 3, the lifting platform 300 may further include a shielding member 306 for a component to be repaired. The shielding member 306 for a component to be repaired covers or shields at least a part of a component to be repaired of the lifting platform 300. The lock status switching member 305 is configured to release covering or shielding of the component to be repaired by the shielding member 306 for a component to be repaired.

Based on the foregoing structure, the shielding member 306 for a component to be repaired is configured to cover or shield the component to be repaired, wherein the covering or shielding of the component to be repaired by the shielding member 306 for a component to be repaired needs to be released by the lock status switching member 305. That is, if the user wants to repair the component to be repaired, the covering or shielding of the component to be repaired by the shielding member 306 for a component to be repaired needs to be released first. If it is desired to release the covering or shielding, the lock status switching member 305 needs to be removed from the locking assembly 304, and as described above, after the lock status switching member 305 is removed, the locking assembly 304 locks the lifting platform 300, to avoid unexpected displacement or lifting of the support table 302. Therefore, by using the foregoing structure, the lifting platform 300 can be reliably locked before the component to be repaired is repaired, thereby realizing a poka-yoke function, providing reliable safety protection, and effectively avoiding safety problems.

In some embodiments, as shown in FIG. 3, the component to be repaired of the lifting platform 300 may be a drive assembly 307 for driving the support assembly 303 to move. Therefore, before the drive assembly 307 is repaired or replaced, it can be ensured that the lifting platform 300 is in a locked state. Therefore, the support table 302 does not suddenly drop due to repair or removal of the drive assembly 307. The present application is not limited thereto, and the component to be repaired may alternatively be another component of the lifting platform 300. For example, the drive assembly 307 is a cylinder, a hydraulic cylinder, a linear actuator, or a ball screw.

As shown in FIG. 3, the support assembly 303 may be in a form of a scissor arm. For example, the support assembly 303 includes a first arm 3031 and a second arm 3032 that are disposed crosswise, and a shaft 3033 that is connected to the first arm 3031 and the second arm 3032. The first arm 3031 and the second arm 3032 may rotate around the shaft 3033.

Upper ends of the first arm 3031 and the second arm 3032 are connected to the support table 302, and lower ends of the first arm 3031 and the second arm 3032 are connected to the base 301 and the drive assembly 307. The drive assembly 307 drives the lower end of at least one of the first arm 3031 and the second arm 3032 to move, and adjusts the distance between the lower end of the first arm 3031 and the lower end of the second arm 3032 to adjust the height of the upper end of the first arm 3031 and the height of the upper end of the second arm 3032, thereby adjusting the height of the support table 302.

The foregoing description is merely an example of a manner in which the drive assembly 307 drives the support assembly 303. The drive assembly 307 may alternatively drive the support assembly 303 in another manner. This is not specifically limited in the present application.

In some embodiments, the shielding member 306 for a component to be repaired may be of various structures capable of covering or shielding the component to be repaired. For example, the shielding member 306 for a component to be repaired may be a housing that can reliably cover or shield the component to be repaired.

FIG. 4 is a schematic diagram of a local structure of a lifting platform according to an embodiment of the present application. In some embodiments, as shown in FIG. 4, the lifting platform 300 may further include a fastening member 308. The fastening member 308 is connected to the shielding member 306 for a component to be repaired to fasten the shielding member 306 for a component to be repaired to a position covering or shielding at least the part of the component to be repaired.

As shown in FIG. 4, the fastening member 308 may be of various structures capable of fastening the shielding member 306 for a component to be repaired. For example, the fastening member 308 may be a screw, and the shielding member 306 for a component to be repaired may be a housing. A through hole that matches the screw is disposed on the housing, and a hole portion that matches the screw is disposed on a specified position of the base 301. The screw is disposed in the hole portion of the base 301 via the through hole, so that the shielding member 306 for a component to be repaired can be fastened to the position that covers or shields at least the part of the component to be repaired. The present application is not limited thereto, and the fastening member 308 may alternatively fasten the shielding member 306 for a component to be repaired in another manner.

In some embodiments, the lock status switching member 305 is configured to release the connection between the fastening member 308 and the shielding member 306 for a component to be repaired. For example, the lock status switching member 305 may be a tool that matches the fastening member 308. For example, the fastening member 308 is a screw, and the lock status switching member 305 is a wrench.

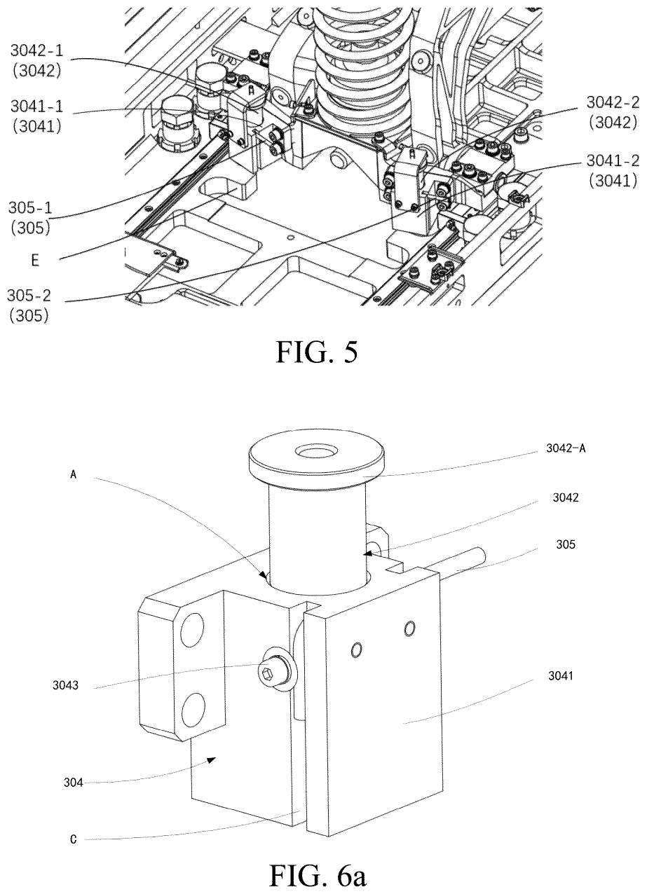

FIG. 5 is another schematic diagram of a local structure of a lifting platform according to an embodiment of the present application. In some embodiments, as shown in FIG. 4 and FIG. 5, there may be one or a plurality of lock status switching members 305 (shown in FIG. 5), and there may be one or a plurality of fastening members 308 (shown in FIG. 4). One lock status switching member 305 may correspond to one fastening member 308 (briefly referred to as one-to-one), that is, the one lock status switching member 305 is sufficient to release a connection between the corresponding fastening member 308 and the shielding member 306 for a component to be repaired. Alternatively, a plurality of lock status switching members 305 may correspond to one fastening member 308 (briefly referred to as many-to-one), that is, a connection between the corresponding fastening member 308 and the shielding member 306 for a component to be repaired can be released only when the plurality of lock status switching members 305 work together. Alternatively, one lock status switching member 305 may correspond to a plurality of fastening members 308 (briefly referred to as one-to-many), that is, connections between the plurality of corresponding fastening members 308 and the shielding member 306 for a component to be repaired can be released by using the one lock status switching member 305. Alternatively, a plurality of lock status switching members 305 may correspond to a plurality of fastening members 308 (briefly referred to as many-to-many), that is, connections between the plurality of corresponding fastening members 308 and the shielding member 306 for a component to be repaired can be released when the plurality of lock status switching members 305 work together.

In some embodiments, as shown in FIG. 5, the locking assembly 304 may include one or a plurality of blocking members 3042. In a case in which the locking assembly 304 includes a plurality of blocking members 3042 and there are a plurality of lock status switching members 305, when one lock status switching member 305 is removed from the locking assembly 304, at least one blocking member 3042 moves to the first position abutting against the base 301. Therefore, when the lock status switching member 305 is removed, the locking assembly 304 can reliably abut against the base 301, so that the lifting platform 300 can be reliably locked.

The following uses a specific example to describe a correspondence among the plurality of blocking members 3042, the plurality of lock status switching members 305, and the plurality of fastening members 308. As shown in FIG. 5, the lock status switching member 305 includes a first lock status switching member 305-1 and a second lock status switching member 305-2. As shown in FIG. 4, the fastening member 308 includes a first fastening member 308-1 and a second fastening member 308-2. The first lock status switching member 305-1 is configured to release a connection between the first fastening member 308-1 and the shielding member 306 for a component to be repaired, and the second lock status switching member 305-2 is configured to release a connection between the second fastening member 308-2 and the shielding member 306 for a component to be repaired. That is, the covering or shielding of the component to be repaired by the shielding member 306 for a component to be repaired can be released only after the first fastening member 308-1 and the second fastening member 308-2 are detached from the first lock status switching member 305-1 and the second lock status switching member 305-2, respectively. Therefore, erroneous detachment of the shielding member 306 for a component to be repaired can be avoided.

As shown in FIG. 5, the locking assembly 304 includes a first blocking member 3042-1 and a second blocking member 3042-2. The first blocking member 3042-1 is movably mounted on a first body member 3041-1, and the second blocking member 3042-2 is movably mounted on a second body member 3041-2. When the first lock status switching member 305-1 is removed from the locking assembly 304, the first blocking member 304-1 abuts against the base 301, and when the second lock status switching member 305-2 is removed from the locking assembly 304, the second blocking member 304-2 abuts against the base 301.

In some embodiments, the first fastening member 308-1 and the second fastening member 308-2 may be screws having different head diameters, for example, the first fastening member 308-1 may be a screw having a head diameter of 4 mm, and the second fastening member 308-2 may be a screw having a head diameter of 5 mm. The first lock status switching member 305-1 and the second lock status switching member 305-2 are respectively wrenches that match the sizes of the first fastening member 308-1 and the second fastening member 308-2.

In some embodiments, the first body member 3041-1 and the second body member 3041-2 are respectively connected to different positions of the lower end of the support assembly 303, for example, to both sides in a direction (for example, a front-rear direction) perpendicular to a moving direction of the lower end of the support assembly 303. Therefore, the support assembly 303 can be prevented from moving on both sides of the support assembly 303, thereby improving locking reliability.

As shown in FIG. 5, a plurality of grooves E may be disposed on the base 301 in the moving direction of the lower end of the support assembly 303, and the blocking member 304 may be engaged with any groove E. Therefore, the lifting platform 300 can be locked by the locking assembly 304 regardless of a position of the lower end of the support assembly 303.

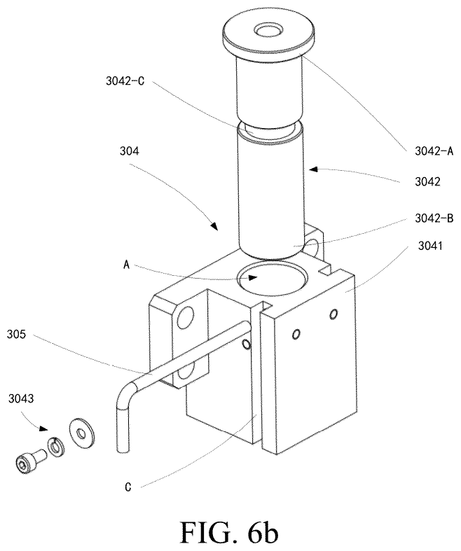

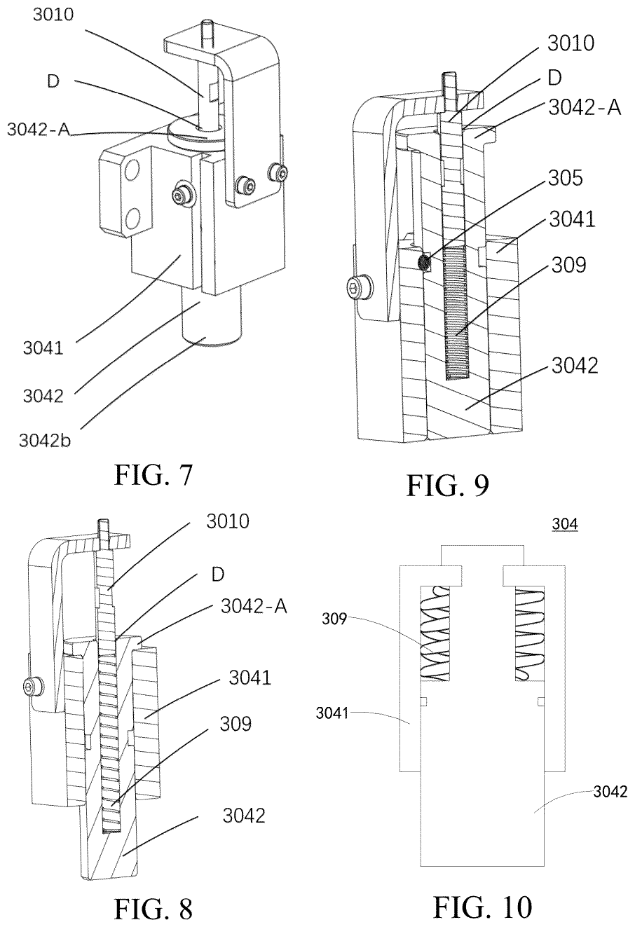

An exemplary description of a structure of the locking assembly is provided below with reference to the accompanying drawings. FIG. 6a and FIG. 6b are schematic diagrams of a locking assembly and a lock status switching member according to an embodiment of the present application. FIG. 7 is a schematic diagram of a locking assembly according to an embodiment of the present application. In some embodiments, as shown in FIG. 6a and FIG. 6b, a through hole A is disposed in the body member 3041 of the locking assembly 304, and the through hole A extends in a moving direction of the blocking member 3042 for accommodating at least a part of the blocking member 3042.

In some embodiments, when the lock status switching member 305 is connected to the locking assembly 304, the blocking member 3042 moves to a second position that does not abut against the base 301. As shown in FIG. 6a, for example, the second position is a position where the lower end (a second end) of the blocking member 3042 is accommodated in the through hole A.

Accordingly, as shown in FIG. 7, the first position where the blocking member 3042 abuts against the base 301 may be a position where the lower end (the second end) 3042-B of the blocking member 3042 is exposed from the body member 3041. At the first position, the upper end (a first end) 3042-A of the blocking member 3042 may abut against the body member 3041 to prevent the blocking member 3042 from sliding out of the through hole A. The present application is not limited thereto, and the blocking member 3042 may alternatively be prevented from sliding out of the through hole A by using another structure.

In some embodiments, as shown in FIG. 6a and FIG. 6b, when the lock status switching member 305 is connected to the locking assembly 304, the lock status switching member 305 is engaged with the blocking member 3042. Therefore, the blocking member 3042 can be held at the second position that does not abut against the base 301.

In some embodiments, as shown in FIG. 6b, the blocking member 3042 may include an engagement portion 3042-C that is engaged with the lock status switching member 305. The engagement portion 3042-C may be a groove disposed on a surface of the blocking member 3042 or a hole portion disposed in the blocking member 3042.

Taking an example in which the engagement portion 3042-C is a groove, the groove may extend in a circumferential direction of the blocking member 3042. For example, the groove may be disposed within the entire circumference of the blocking member 3042. Therefore, regardless of a spin angle at which the blocking member 3042 is accommodated in the through hole A, a part of the groove can be engaged with the lock status switching member 305, which facilitates mounting and manufacturing of the blocking member 3042.

The present application is not limited thereto, and the groove may alternatively be disposed within a partial circumference of the blocking member 3042. In this case, the blocking member 3042 needs to be accommodated in the through hole A at a specified spin angle, so that the groove is located at a position that can be engaged with the lock status switching member 305.

In some embodiments, a marker may be disposed on the blocking member 3042 to mark the spin angle of the blocking member 3042, or both the blocking member 3042 and the through hole A may be disposed in a non-axisymmetric shape to ensure that the blocking member 3042 is always accommodated in the through hole A at a specified spin angle.

A position of the groove in a vertical direction of the blocking member 3042 may be as follows. When the lock status switching member 305 is engaged with the groove, the lower end of the blocking member 3042 does not abut against the base 301.

Taking an example in which the engagement portion 3042-C is a hole portion, the hole portion may be a through hole that runs through the blocking member 3042, provided that an extending direction of the through hole is different from the moving direction of the blocking member 3042. For example, the extending direction of the through hole is perpendicular to the moving direction of the blocking member 3042. The present application is not limited thereto, and the hole portion may alternatively be a hole with a bottom that is disposed in the blocking member 3042.

When the engagement portion 3042-C is a hole portion, the blocking member 3042 needs to be accommodated in the through hole A at a specified spin angle, so that the hole portion is located at a position that can be engaged with the lock status switching member 305. Similar to the form in which the engagement portion 3042-C is a groove, the spin angle of the blocking member 3042 may be marked or ensured in the foregoing manner.

A position of the hole portion in the vertical direction of the blocking member 3042 may be as follows. When the lock status switching member 305 is engaged with the hole portion, the lower end of the blocking member 3042 does not abut against the base 301.

In some embodiments, a second hole portion may be disposed in the body member 3041, and the second hole portion extends in a direction different from the moving direction of the blocking member 3042 for accommodating the lock status switching member 305.

When the lock status switching member 305 is located in the second hole portion, and the blocking member 3042 is located at a second position, the lock status switching member 305 is engaged with the blocking member 3042.

The present application is not limited thereto, and the second hole portion may alternatively not be disposed in the body member 3041. For example, when the lock status switching member 305 abuts against an end portion (for example, an upper end) that is of the body member 3041 and that is away from the base, and the blocking member 3042 is located at the second position, the lock status switching member 305 is engaged with the blocking member 3042. That is, in a state in which the lock status switching member 305 is located on the upper end face of the body member 3041, the lock status switching member 305 is engaged with the blocking member 3042 to hold the blocking member 3042 at the second position that does not abut against the base 301.

In some embodiments, as shown in FIG. 6a and FIG. 6b, a groove C may be disposed on a side surface of the body member 3041, and the groove C may be configured to accommodate a part that is of the lock status switching member 305 and that is located outside the second hole portion to avoid interference between the lock status switching member 305 and another component of the lifting platform 300. For example, the lock status switching member 305 is an L-shaped wrench. A first rod of the L-shaped wrench is accommodated in the second hole portion, and a second rod of the L-shaped wrench is accommodated in the groove C.

In some embodiments, as shown in FIG. 6a and FIG. 6b, the locking assembly 304 may further include a limiting portion 3043. When the lock status switching member 305 is connected to the locking assembly 304, the limiting portion 3043 is configured to limit the lock status switching member 305 to prevent the lock status switching member 305 from sliding out of the locking assembly 304.

As shown in FIG. 6a and FIG. 6b, the limiting portion 3043 may be disposed at an edge of the groove C with at least a part thereof covering an opening of the groove C, so that the lock status switching member 305 can be reliably prevented from sliding out.

The limiting portion 3043 may be in various forms, for example, the limiting portion 4043 may include a screw and a baffle plate. The screw is installed at the edge of the groove C through a through hole of the baffle plate, and at least a part of the baffle plate extends to the opening of the groove C.

The present application is not limited thereto, and the locking assembly 304 may alternatively not include a limiting portion. For example, mating threads or the like may be disposed on the lock status switching member 305 and the engagement portion 3042-C of the blocking member 3042, so that the lock status switching member 305 can be prevented from sliding out via the threads.

In some embodiments, in order to enable the blocking member 3041 to reliably abut against the base 301, an elastic member may be disposed in the locking assembly 304. The elastic member may be accommodated in the through hole A, and is configured to apply a force to the blocking member 3041 to move the blocking member 3041 toward the base 301.

FIG. 8 is another schematic diagram of a locking assembly according to an embodiment of the present application. FIG. 9 is another schematic diagram of a locking assembly and a lock status switching member according to an embodiment of the present application. FIG. 8 and FIG. 9 are cross-sectional views of the elastic member 309 in different states. In some embodiments, as shown in FIG. 8 and FIG. 9, the elastic member 309 is disposed inside the blocking member 3042.

As shown in FIG. 7 to FIG. 9, a first hole portion D is disposed on a first end 3042-A that is of the blocking member 3042 and that is away from the base 301. The locking assembly 304 further includes a guide member 3010. One end (for example, an upper end) of the guide member 3010 is fixedly connected to the body member 3041, the other end (for example, a lower end) of the guide member 3010 is located in the first hole portion D, and the blocking member 3042 can move along the guide member 3010.

As shown in FIG. 8 and FIG. 9, the elastic member 309 is accommodated in the first hole portion D, one end (for example, a lower end) of the elastic member 309 abuts against the bottom of the first hole portion D, and the other end (for example, an upper end) of the elastic member 309 abuts against the guide member 3010. Therefore, when the lock status switching member 305 is engaged with the blocking member 3042, the blocking member 3042 is located at the second position, the distance between the first hole portion D and the guide member 3010 is reduced, and the elastic member 309 is in a first compressed state. After the lock status switching member 305 is removed from the locking assembly 304, the blocking member 3042 moves from the second position to the first position under the action of the elastic member 309 and gravity, the distance between the first hole portion D and the guide member 3010 is increased, and the elastic member 309 is in a natural state or a second compressed state, wherein a degree of compression in the second compressed state is lower than that in the first compressed state.

A structure in which the elastic member 309 is disposed inside the blocking member 3042 is described above in FIG. 8 and FIG. 9. The present application is not limited thereto, and the elastic member 309 may alternatively be disposed on a peripheral side of the blocking member 3042.

FIG. 10 is a schematic diagram of a locking assembly according to an embodiment of the present application. As shown in FIG. 10, the elastic member 309 is disposed on the peripheral side of the blocking member 3042. An upper end of the elastic member 309 abuts against the body member 3041, and a lower end of the elastic member 309 abuts against the blocking member 3042. Therefore, when the lock status switching member 305 is engaged with the blocking member 3042, the blocking member 3042 is located at the second position, and the elastic member 309 is in a third compressed state. After the lock status switching member 305 is removed from the locking assembly 304, the blocking member 3042 moves from the second position to the first position under the action of the elastic member 309 and gravity, and the elastic member 309 is in a natural state or a fourth compressed state, wherein a degree of compression in the fourth compressed state is lower than that in the third compressed state.

It is worth noting that only the individual components or modules relevant to this embodiment have been described above, but the present application is not limited thereto. The lifting platform may further include other components or modules, and reference may be made to the related art for details of these components or modules.

According to the foregoing embodiment, a locking assembly capable of preventing movement of a support assembly of a lifting platform, and a lock status switching member detachably connected to the locking assembly are disposed in the lifting platform, and the locking assembly prevents the movement of the support assembly when the lock status switching member is removed from the locking assembly. Therefore, a user only needs to remove the lock status switching member from the locking assembly to lock the lifting platform, that is, the lifting platform is locked by a removing operation instead of a mounting operation. Therefore, this can simplify the locking operation, and help avoid safety problems.

An embodiment of the present application further provides a medical device. The medical device includes the lifting platform as described above. As shown in FIG. 1, the medical device may be a CT system 100 or the like.

According to the foregoing embodiment, in the lifting platform of the medical device, a locking assembly capable of preventing movement of a support assembly of the lifting platform, and a lock status switching member detachably connected to the locking assembly are disposed, and the locking assembly prevents the movement of the support assembly when the lock status switching member is removed from the locking assembly. Therefore, a user only needs to remove the lock status switching member from the locking assembly to lock the lifting platform, that is, the lifting platform is locked by a removing operation instead of a mounting operation. Therefore, this can simplify the locking operation, and help avoid safety problems.

The lifting platform is disposed in the medical device, so that the operation can be simplified during the operation of the medical device, and safety problems can be avoided.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

The present application is described above with reference to specific implementations. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application. As used herein, the term “example” represents serving as a non-limiting example, instance, or illustration. As used herein, the term “for example” refers to a list of one or a plurality of non-limiting examples, instances, or illustrations.

Preferred implementations of the present application are described above with reference to the accompanying drawings. Many features and advantages of the implementations are clear according to the detailed description. Therefore, the appended claims are intended to cover all these features and advantages that fall within the true spirit and scope of these implementations. In addition, as many modifications and changes could be easily conceived of by those skilled in the art, the implementations of the present application are not limited to the illustrated and described precise structures and operations, but can encompass all appropriate modifications, changes, and equivalents that fall within the scope of the implementations.

1. A lifting platform, characterized by comprising:
a base;
a support table disposed on an upper side of the base;
a support assembly connecting the support table and the base and driving the support table to lift;
a locking assembly including a body member connected to the support assembly and a blocking member movably mounted on the body member, wherein the blocking member prevents movement of the support assembly when moving to a first position abutting against the base; and
a lock status switching member detachably connected to the locking assembly, wherein the blocking member moves to the first position when the lock status switching member is removed from the locking assembly.

2. The lifting platform according to claim 1, further including:
a shielding member for a component to be repaired covering or shielding at least a part of a component to be repaired of the lifting platform,
wherein the lock status switching member is configured to release covering or shielding of the component to be repaired by the shielding member for a component to be repaired.

3. The lifting platform according to claim 2, further including:
a fastening member connected to the shielding member for a component to be repaired to fasten the shielding member for a component to be repaired to a position covering or shielding at least the part of the component to be repaired,
wherein the lock status switching member is configured to release the connection between the fastening member and the shielding member for a component to be repaired.

4. The lifting platform according to claim 3, wherein
the lock status switching member comprises a first lock status switching member and a second lock status switching member;
the fastening member comprises a first fastening member and a second fastening member; and
the first lock status switching member is configured to release a connection between the first fastening member and the shielding member for a component to be repaired, and the second lock status switching member is configured to release a connection between the second fastening member and the shielding member for a component to be repaired.

5. The lifting platform according to claim 4, wherein
the blocking member includes a first blocking member and a second blocking member; and
the first blocking member abuts against the base when the first lock status switching member is removed from the locking assembly, and the second blocking member abuts against the base when the second lock status switching member is removed from the locking assembly.

6. The lifting platform according to claim 1, wherein
a through hole is disposed in the body member, and the through hole extends in a moving direction of the blocking member for accommodating at least a part of the blocking member.

7. The lifting platform according to claim 6, wherein the locking assembly further includes:
an elastic member that is accommodated in the through hole and that applies a force to the blocking member to move the blocking member toward the base.

8. The lifting platform according to claim 7, wherein
the elastic member is disposed inside the blocking member.

9. The lifting platform according to claim 8, wherein
a first hole portion is disposed on a first end that is of the blocking member and that is away from the base;
the locking assembly further includes a guide member, wherein one end of the guide member is fixedly connected to the body member, the other end of the guide member is located in the first hole portion, and the blocking member moves along the guide member; and
the elastic member is accommodated in the first hole portion, wherein one end of the elastic member abuts against the bottom of the first hole portion, and the other end of the elastic member abuts against the guide member.

10. The lifting platform according to claim 1, wherein
when the lock status switching member is connected to the locking assembly, the blocking member moves to a second position that does not abut against the base.

11. The lifting platform according to claim 1, wherein
when the lock status switching member is connected to the locking assembly, the lock status switching member is engaged with the blocking member.

12. The lifting platform according to claim 11, wherein
the blocking member comprises an engagement portion that is engaged with the lock status switching member, and the engagement portion is a groove disposed on a surface of the blocking member or a hole portion disposed in the blocking member.

13. The lifting platform according to claim 11, wherein
a second hole portion is disposed in the body member, and the second hole portion extends in a direction different from a moving direction of the blocking member for accommodating the lock status switching member; and
when the lock status switching member is located in the second hole portion, and the blocking member is located at a second position, the lock status switching member is engaged with the blocking member.

14. The lifting platform according to claim 11, wherein
when the lock status switching member abuts against an end portion that is of the body member and that is away from the base, and the blocking member is located at a second position, the lock status switching member is engaged with the blocking member.
